(19) United States
(12) Patent Application Publication
(10) Pub. No.: US 2024/0230641 A1
(43) Pub. Date: Jul. 11, 2024

(54) Methods of Identifying Active Lupus Nephritis Flare

(71) Applicant: Beth Israel Deaconess Medical Center, Inc., Boston, MA (US)

(72) Inventors: George C. Tsokos, Boston, MA (US); Rhea Bhargava, Boston, MA (US)

(21) Appl. No.: 18/550,008

(22) PCT Filed: Mar. 11, 2022
(86) PCT No.: PCT/US2022/071088

§ 371 Date: Sep. 11, 2023

Related U.S. Application Data
(60) Provisional application No. 63/200,500, filed on Mar. 11, 2021.

(51) Int. Cl. G01N 33/564; C12Q 1/6851; C12Q 1/6883; A61B 10/00

(52) U.S. Cl. CPC G01N 33/564; C12Q 1/6851; C12Q 1/6883; A61B 10/007; C12Q 2600/158; G01N 2333/54; G01N 2333/912; G01N 2800/104; G01N 2800/347

(57) ABSTRACT

Provided herein are methods of identifying and/or diagnosing an active lupus nephritis (LN) flare in a subject, the method comprising obtaining a sample from the subject; isolating cells from the urine sample; and determining a level of expression of calcium/calmodulin dependent protein kinase IV (CaMK4), IL-23, IL-17 receptor, and/or arginase 1, wherein an increased level of expression of CaMK4, IL-23, and/or IL-17 receptor as compared to a control or a decreased level of expression of arginase 1 as compared to a control indicates an active lupus nephritis (LN) flare in the subject.

### STATEMENT REGARDING FEDERALLY FUNDED RESEARCH

This invention was made with government support under Grant No. RO1AR064350 awarded by the Nation Institutes of Health. The government has certain rights in the invention.

### CROSS REFERENCE TO RELATED APPLICATION

This invention claims priority to U.S. Provisional Application No. 63,200,500, filed Mar. 11, 202, the disclosure of which is incorporated herein by reference in its entirety.

### BACKGROUND OF THE INVENTION

Lupus nephritis (LN) represents one of the most serious complications of systemic lupus erythematosus (SLE) and is associated with significant morbidity and mortality. People with LN have a higher standardized mortality ratio compared with those without LN (6-6.8 versus 2.4-3) (1-4) and up to 30% of them develop end stage renal disease (5), which is also a significant predictor of poor outcomes (6-10). Achievement and maintenance of remission of LN improves the 10-year survival from 46% to 95% (11).

The pathogenesis of LN involves the deposition of circulating or in situ formed immune complexes in different areas of the glomerulus leading to activation of components of the innate and adaptive immune system (12-16). However, the exact immunopathogenesis of LN is still elusive and the inciting events that lead to resident kidney cell injury and organ failure are largely unknown. It has been proposed that podocyte injury occurs early in LN after deposition of immune complexes and precedes irreversible glomerular damage (17-19). Depletion of more than 30% of podocytes causes glomerular destabilization which leads to eventual glomerulosclerosis and has been associated with the severity of LN (20-21).

Calcium signaling plays a key role in the maintenance of the actin cytoskeleton of cells (22-28). It has been previously demonstrated that calcium/calmodulin kinase IV (CaMK4) expression is increased in podocytes of lupus-prone MRL/lpr mice and that exposure of cultured human podocytes to IgG from individuals with SLE leads to CaMK4 upregulation (19, 29). Genetic or pharmacologic inhibition of CaMK4 prevents development of nephritis in lupus-prone mice (29-31). Interestingly, inhibition of CaMK4 in podocytes only, prevents immune complex deposition and preserves renal function despite systemic autoimmunity remaining intact (19).

The significant morbidity and mortality associated with LN highlights the importance of identifying patients with SLE who are likely to develop kidney disease and can benefit from early therapeutic intervention. Demonstrated herein, it was shown that overexpression of CaMK4 in renal biopsy material represents a biomarker of active LN. More importantly, IgG from individuals with LN, but not from those with SLE without clinical evidence of LN, upregulates CaMK4 expression in cultured podocytes. In parallel, podocytes present in the urine of individuals with active LN display increased CaMK4 levels. Mechanistically, it was demonstrated that the presence of fucose moieties in combination with under-galactosylated N-glycans on IgG are responsible for the increased expression of CaMK4 in podocytes. CaMK4 represses nephrin transcription through a signaling pathway that involves CaMK4-induced phosphorylation of NFκB and upregulation of SNAIL, the transcriptional repressor of nephrin. The results provided herein provide new approaches and tools that could limit the need for kidney biopsies in diagnosing LN, and propose the consideration of glycosylation modulators to prevent or reverse the development of LN.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

In one general aspect, the invention relates to methods for identifying and/or diagnosing an active lupus nephritis (LN) flare in a subject. The methods comprise (a) obtaining a sample from the subject; (b) isolating cells from the sample; and (c) determining a level of expression of calcium/calmodulin dependent protein kinase IV (CaMK4), IL-23, IL-17 receptor, or arginase 1 from the isolated cells, wherein an increased level of expression of CaMK4, IL-23, and/or IL-17 receptor as compared to a control or a decreased level of expression of arginase 1 as compared to a control indicates an active lupus nephritis (LN) flare in the subject.

Also provided are methods of monitoring the response to the treatment of a subject with an active lupus nephritis (LN) flare. The methods comprise (a) obtaining a sample from the subject undergoing treatment for an active LN flare; (b) isolating cells from the sample; and (c) determining a level of expression of calcium/calmodulin dependent protein kinase IV (CaMK4), IL-23, IL-17 receptor, or arginase 1 from the isolated cells, wherein an decreased level of expression of CaMK4, IL-23, and/or IL-17 receptor as compared to a control or an increased level of expression of arginase 1 as compared to a control indicates an effective response to the treatment of the active LN flare in the subject.

In certain embodiments, the sample is selected from a tissue sample or a urine sample. In certain embodiments, the isolated cells are selected from podocytes or tubular epithelial cells. In certain embodiments, the sample is a urine sample and the isolated cells are podocytes. The podocytes can, for example, be isolated from the urine sample by a podocyte specific antibody. In certain embodiments, the podocyte specific antibody is selected from an anti-nephrin antibody, an anti-podocin antibody, and/or an anti-synaptopodin antibody.

In certain embodiments, the level of expression of CaMK4, IL-23, IL-17 receptor, and/or arginase is determined using a reverse transcriptase-polymerase chain reaction (RT-PCR), a quantitative RT-PCR (qRT-PCR) assay, a northern blot assay, a microarray assay, a flow cytometry assay, an immunofluorescence assay, and/or a western blot assay.

Also provided are methods for identifying and/or diagnosing an active lupus nephritis (LN) flare in a subject. The methods comprise (a) obtaining a urine sample from the subject; (b) isolating podocytes from the urine sample; and (c) determining a level of isolated podocytes, wherein an increased level of podocytes as compared to a control indicates an active lupus nephritis (LN) flare. In certain embodiments, the methods comprise (d) determining a level of expression of CaMK4 in the isolated urine podocytes, wherein an increased level of expression further indicates an active lupus nephritis (LN) flare.

In certain embodiments, the podocytes are isolated from the urine sample by a podocyte specific antibody. The podocyte specific antibody can, for example, be selected from an anti-nephrin antibody, an anti-podocin antibody, and/or an anti-synaptopodin antibody.

Also provided are methods of identifying an active lupus nephritis (LN) flare in a subject. The methods comprise (a) culturing a podocyte cell line; (b) obtaining serum IgG from the subject; (c) contacting the podocyte cell line with the serum IgG; and (d) determining a level of expression of calcium/calmodulin dependent protein kinase IV (CaMK4), IL-17 receptor, IL-23, and/or CD86 in the IgG-contacted podocyte cell line, wherein an increase in the level of expression of CaMK4, IL-17 receptor, IL-23, and/or CD86 as compared to a control indicates an active lupus nephritis (LN) flare in the subject.

Also provided are methods of monitoring the response to the treatment of a subject with an active lupus nephritis (LN) flare. The methods comprise (a) culturing a podocyte cell line; (b) obtaining serum IgG from the subject undergoing treatment for an active LN flare; (c) contacting the podocyte cell line with the serum IgG; and (d) determining a level of expression of calcium/calmodulin dependent protein kinase IV (CaMK4), IL-17 receptor, IL-23, and/or CD86 in the IgG-contacted podocyte cell line, wherein a decrease in the level of expression of CaMK4, IL-17 receptor, IL-23, and/or CD86 as compared to a control indicates an effective response to the treatment of the active LN flare in the subject.

In certain embodiments, the level of expression of CaMK4, IL-17 receptor, IL-23, and/or CD86 is determined using a reverse transcriptase-polymerase chain reaction (RT-PCR), a quantitative RT-PCR (qRT-PCR) assay, a northern blot assay, a microarray assay, and/or a western blot assay.

In certain embodiments, the methods further comprise determining a level of CD80, tumor necrosis factor-alpha (TNF-α), and/or IL-6, wherein an increase in the level of expression of CaMK4, IL-17 receptor, IL-23, and/or CD86 and no change in the level of expression of CD80, TNF-α, and/or IL-6 as compared to a control further indicates an active LN flare in the subject.

In certain embodiments, the methods further comprise determining a level of CD80, tumor necrosis factor-alpha (TNF-α), and/or IL-6, wherein a decrease in the level of expression of CaMK4, IL-17 receptor, IL-23, and/or CD86 and no change in the level of expression of CD80, TNF-α, and/or IL-6 as compared to a control further indicates an effective response to the treatment of the active LN flare in the subject.

The level of expression of CD80, TNF-α, and/or CD86 can, for example, be determined using a reverse transcriptase-polymerase chain reaction (RT-PCR), a quantitative RT-PCR (qRT-PCR) assay, a northern blot assay, a microarray assay, and/or a western blot assay.

In certain embodiments, the subject has a disease selected from glomerulonephritis, anti-neutrophilic cytoplasmic autoantibody (ANCA) vasculitis, or systemic lupus erythematosus (SLE).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-IC show IgG from patients with LN upregulated CaMK4 in podocytes in contrast to IgG from individuals with SLE without LN and healthy controls. (FIG. 1A) CaMK4 mRNA and (FIG. 1B) CaMK4 protein expression (representative experiment) in podocytes after exposure to IgG from lupus (L) patients with no kidney involvement, active lupus nephritis (LN) and healthy controls (H). (FIG. 1C) Densitometric quantification of Western blot data from 3 independent experiments (n=5 individuals per group).

FIGS. 2A-2E show treatment of IgG with α-fucosidase prevented the upregulation of CAMK4 in podocytes. (FIG. 2A) Both deglycosylated (Degly) and untreated IgG from individuals with LN colocalized with neonatal Fc receptor (FcRn) in human podocytes (20×). (FIG. 2B) Intensity of Alexa Fluor tagged PNGase F-treated (deglycosylated) and untreated LN-derived IgG in podocytes was analyzed by immunofluorescence staining (20×). (FIG. 2C) CAMK4 expression was evaluated after exposure of podocytes to deglycosylated or untreated IgG from healthy controls (H) or patients with LN (a representative experiment is shown). (FIG. 2D) Densitometry was performed for quantification of the results in FIG. 2D (3 independent experiments were performed). Error bars represent mean±SEM. *p<0.05; ****p<0.0001, one-way ANOVA, Bonferroni post-test correction. (FIG. 2E) CAMK4 expression in podocytes after exposure to IgG derived from a patient with LN before (noRx) or after treatment with β-N-acetylglucosaminidase (AC), Neuraninidase A (NA), Neuraminidase S (NS), or α-Fucosidase (F). (FIG. 2F) Densitometry was performed for quantification of the results in FIG. 2E. Error bars represent mean SEM. *p<0.05; ****p<0.0001, one-way ANOVA, Bonferroni post-test correction (3 independent experiments utilizing IgG from 3 different individuals were performed for each representative experiment displayed above).

FIGS. 3A-3D show IgG N-glycome in individuals with active LN was significantly different from healthy individuals and those with SLE without active kidney disease. Mass Spectrometric quantification analysis of IgG from healthy controls (1), SLE patient with no kidney involvement (2), SLE patient with LN in remission (3), and SLE patients with active LN (4 and 5). (FIG. 3A) Fucosylated N glycans (FIG. 3B) Bisected N-glycans (FIG. 3C) Di-undergalactosylated N glycans (FIG. 3D) Mono-undergalactosylated N glycans. (n=5).

FIGS. 4A-4F show IgG from individuals with active LN were under galactosylated compared to IgG from healthy controls and individuals with SLE without kidney disease. Representative images of mass spectrometric analysis of IgG from (FIG. 4A) healthy control, (FIG. 4B) SLE patient with no kidney involvement, (FIG. 4C) SLE patient with LN in remission, and (FIG. 4D and FIG. 4E) SLE patient with active LN. (FIG. 4F) Heat map displaying glycan moieties of different masses for each sample. Red arrow represents the glycan most prevalent in active LN while green arrow indicates the glycan most prevalent in healthy controls (n=5).

FIGS. 5A-5D show the presence of galactose on IgG had a protective role in podocyte injury. (FIG. 5A) CAMK4 expression in podocytes after exposure to untreated IgG and β-galactosidase-treated IgG from healthy controls (H) and individuals with LN. (FIG. 5B) Densitometry was performed for quantification of results in FIG. 5A (FIG. 5C) Nephrin expression in podocytes after exposure to untreated IgG and galactosidase-treated IgG from healthy controls (H) and individuals with LN. (FIG. 5D) Densitometry was performed for quantification of the results in FIG. 5C. Error bars represent mean±SEM. *p<0.05; ****p<0.0001, one-way ANOVA, Bonferroni post-test correction (3 independent experiments utilizing IgG from 3 different individuals were performed for each representative experiment displayed above).

FIGS. 6A-6I show CAMK4 regulated nephrin transcription through NFκb/p65 pathway and SNAIL upregulation. Podocytes were cultured in the presence of IgG from healthy controls or individuals with LN. (FIG. 6A) Nephrin mRNA was decreased in podocytes after exposure to IgG from individuals with LN in a CAMK4-dependent manner. Western blot was utilized to measure the expression of SNAIL protein (FIG. 6B, FIG. 6C), and NFκB p65 (FIG. 6D, FIG. 6E) in the presence or absence of CAMK4 siRNA. (FIG. 6F) Nephrin mRNA and (FIG. 6G) SNAIL mRNA were quantified in podocytes after exposure to IgG from healthy controls and from patients with LN in the presence or absence of NFκB (p65). (FIG. 6H) CAMK4 was immunoprecipitated from podocytes after exposure to IgG from individuals with LN and healthy controls and immunoblotted (IB) with an antibody against p65 (FIG. 6I) Schematic illustration of the proposed mechanism whereby CAMK4 phosphorylates and activates NFκB p65, which upregulates SNAIL transcription leading to repression of nephrin transcription.

FIGS. 7A-7B show individuals with active LN had increased CAMK4 expression in podocytes. (FIG. 7A) Representative images of immunofluorescence staining for synaptopodin and CAMK4 in glomeruli from kidney biopsies of patients with LN and controls without any glomerular lesion. Blue: DAPI, Red: Synaptopodin, Green: CAMK4. (FIG. 7B) Representative images of immunofluorescence staining for nephrin in glomeruli from kidney biopsies of patients with LN and controls without any glomerular lesion. Green: Nephrin. Blue: DAPI (n=30 patients with LN and 5 controls without any glomerular lesion).

FIGS. 8A-8D show CAMK4 expression in urine podocytes identified individuals with active LN. (FIG. 8A) Total urine podocin mRNA in individuals with SLE without nephritis (no LN), with active lupus nephritis (LN) and with LN who showed clinical response to treatment (responder). (FIG. 8B) Representative images of immunofluorescence staining for synaptopodin (green) and CAMK4 (red) in urine cells from an individual with SLE and no LN, an individual with LN and a responder. (FIG. 8C) Total urine CAMK4 mRNA and (FIG. 8D) Urine podocyte CAMK4 mRNA in individuals with SLE without nephritis (no LN) (n=4), individuals with LN (n=6) and responders (n=5).

FIGS. 9A-9F show IgG from patients with lupus nephritis (LN) caused upregulation of different genes in podocytes compared to those with SLE without kidney disease (L) and healthy controls (H). (FIG. 9A) IL-17r; (FIG. 9B) IL-23A; (FIG. 9C) CD86; (FIG. 9D) CD80; (FIG. 9E) TNF-alpha; (FIG. 9F) IL-6 mRNA expression in podocytes after exposure to IgG from SLE patients with no kidney involvement (L), active LN (LN) and healthy controls (H) (n=15).

FIG. 10 shows mass spectrometric quantification analysis of sialylated N glycans on IgG from healthy controls (1), SLE patient with no kidney involvement (2), SLE patient with LN in remission (3), and SLE patient with active LN (4 and 5). (n=5).

FIG. 11 shows SNAIL mRNA was increased in podocytes after exposure to IgG from individuals with LN compared to healthy controls.

FIG. 12 shows CAMK4 was immunoprecipitated from podocytes after exposure to IgG from individuals with LN and healthy controls and immunoblotted (IB) with an antibody against p50.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, “subject” or “patient” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” is intended to include any sampling of cells, tissues, or bodily fluids. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art.

Methods of Identifying and/or Diagnosing an Active Lupus Nephritis (LN) Flare and/or Monitoring a Response to Treatment of an Active LN Flare.

In one general aspect, provided herein are methods for identifying and/or diagnosing an active lupus nephritis (LN) flare in a subject. The methods comprise (a) obtaining a sample from the subject; (b) isolating cells from the sample; and (c) determining a level of expression of calcium/calmodulin dependent protein kinase IV (CaMK4), IL-23, IL-17 receptor, and/or arginase 1, wherein an increased level of expression of CaMK4, IL-23, and/or IL-17 receptor as compared to a control or a decreased level of arginase 1 as compared to a control indicates an active lupus nephritis (LN) flare in the subject.

As used herein for methods of identifying and/or a diagnosing an active LN flare in a subject, a control can, for example, include a sample from a different subject without an active LN flare and/or a sample from the same subject prior to the active LN flare. A person skilled in the art would understand the proper control for the methods for identifying and/or diagnosing an active LN flare in a subject.

Also provided are methods of monitoring the response to the treatment of a subject with an active lupus nephritis (LN) flare. The methods comprise (a) obtaining a sample from the subject undergoing treatment for an active LN flare; (b) isolating cells from the sample; and (c) determining a level of expression of calcium/calmodulin dependent protein kinase IV (CaMK4), IL-23, IL-17 receptor, or arginase 1, wherein an decreased level of expression of CaMK4, IL-23, and/or IL-17 receptor as compared to a control or an increased level of arginase 1 as compared to a control indicates an effective response to the treatment of the active LN flare in the subject.

As used herein for methods of monitoring the response to the treatment of a subject with an active LN flare, a control can, for example, include a sample from the same subject prior to treatment for the active LN flare and/or a sample from a subject that has not received treatment for an active LN flare.

In certain embodiments, the sample is selected from a tissue sample or a urine sample. In certain embodiments, the isolated cells are selected from podocytes or tubular epithelial cells. In certain embodiments, the sample is a urine sample and the isolated cells are podocytes. The podocytes can, for example, be isolated from the urine sample by a podocyte specific antibody. In certain embodiments, the podocyte specific antibody is selected from an anti-nephrin antibody, an anti-podocin antibody, and/or an anti-synaptopodin antibody.

In certain embodiments, the level of expression of CaMK4, IL-23, IL-17 receptor, and/or arginase is determined using a reverse transcriptase-polymerase chain reaction (RT-PCR), a quantitative RT-PCR (qRT-PCR) assay, a northern blot assay, a microarray assay, a flow cytometry assay, an immunofluorescence assay, and/or a western blot assay.

Also provided are methods for identifying and/or diagnosing an active lupus nephritis (LN) flare in a subject. The methods comprise (a) obtaining a urine sample from the subject; (b) isolating podocytes from the urine sample; and (c) determining a level of isolated podocytes, wherein an increased level of podocytes as compared to a control indicates an active lupus nephritis (LN) flare. In certain embodiments, the methods further comprise (d) determining an expression level of CaMK4 in the isolated urine podocytes, wherein an increased level of expression further indicates an active lupus nephritis (LN) flare.

In certain embodiments, the podocytes are isolated from the urine sample by a podocyte specific antibody. The podocyte specific antibody can, for example, be selected from an anti-nephrin antibody, an anti-podocin antibody, and/or an anti-synaptopodin antibody.

Also provided are methods of identifying and/or diagnosing an active lupus nephritis (LN) flare in a subject. The methods comprise (a) culturing a podocyte cell line; (b) obtaining serum IgG from the subject; (c) contacting the podocyte cell line with the serum IgG; and (d) determining a level of expression of calcium/calmodulin dependent protein kinase IV (CaMK4), IL-17 receptor, IL-23, and/or CD86 in the IgG-contacted podocyte cell line, wherein an increase in the level of expression of CaMK4, IL-17 receptor, IL-23, and/or CD86 as compared to a control indicates an active lupus nephritis (LN) flare in the subject.

As used herein for methods of identifying and/or a diagnosing an active LN flare in a subject, a control can, for example, include a sample from a different subject without an active LN flare and/or a sample from the same subject prior to the active LN flare. A person skilled in the art would understand the proper control for the methods for identifying and/or diagnosing an active LN flare in a subject.

Also provided are methods of monitoring the response to the treatment of a subject with an active lupus nephritis (LN) flare. The methods comprise (a) culturing a podocyte cell line; (b) obtaining serum IgG from the subject undergoing treatment for an active LN flare; (c) contacting the podocyte cell line with the serum IgG; and (d) determining a level of expression of calcium/calmodulin dependent protein kinase IV (CaMK4), IL-17 receptor, IL-23, and/or CD86 in the IgG-contacted podocyte cell line, wherein a decrease in the level of expression of CaMK4, IL-17 receptor, IL-23, and/or CD86 as compared to a control indicates an effective response to the treatment of the active LN flare in the subject.

As used herein for methods of monitoring the response to the treatment of a subject with an active LN flare, a control can, for example, include a sample from the same subject prior to treatment for the active LN flare and/or a sample from a subject that has not received treatment for an active LN flare.

In certain embodiments, the level of expression of CaMK4, IL-17 receptor, IL-23, and/or CD86 is determined using a reverse transcriptase-polymerase chain reaction (RT-PCR), a quantitative RT-PCR (qRT-PCR) assay, a northern blot assay, a microarray assay, and/or a western blot assay.

In certain embodiments, the methods further comprise determining a level of CD80, tumor necrosis factor-alpha (TNF-α), and/or IL-6, wherein an increase in the level of expression of CaMK4, IL-17 receptor, IL-23, and/or CD86 and no change in the level of expression of CD80, TNF-α, and/or IL-6 as compared to a control further indicates an active LN flare in the subject.

In certain embodiments, the methods further comprise determining a level of CD80, tumor necrosis factor-alpha (TNF-α), and/or IL-6, wherein a decrease in the level of expression of CaMK4, IL-17 receptor, IL-23, and/or CD86 and no change in the level of expression of CD80, TNF-α, and/or IL-6 as compared to a control further indicates an effective response to the treatment of the active LN flare in the subject.

The level of expression of CD80, TNF-α, and/or CD86 can, for example, be determined using a reverse transcriptase-polymerase chain reaction (RT-PCR), a quantitative RT-PCR (qRT-PCR) assay, a northern blot assay, a microarray assay, and/or a western blot assay.

In certain embodiments, the subject has a disease selected from glomerulonephritis, anti-neutrophilic cytoplasmic autoantibody (ANCA) vasculitis, or systemic lupus erythematosus (SLE).

### Embodiments

The invention provides also the following non-limiting embodiments.
Embodiment 1 is a method for identifying and/or diagnosing an active lupus nephritis (LN) flare in a subject, the method comprising:
   a. obtaining a sample from the subject;
   b. isolating cells from the sample; and
   c. determining a level of expression of calcium/calmodulin dependent protein kinase IV (CaMK4), IL-23, IL-17 receptor, or arginase 1, wherein an increased level of expression of CaMK4, IL-23, and/or IL-17 receptor as compared to a control or a decreased level of expression of arginase 1 as compared to a control indicates an active lupus nephritis (LN) flare in the subject.
Embodiment 1a is the method of embodiment 1, wherein the sample is selected from a tissue sample or a urine sample.
Embodiment 2 is the method of embodiment 1 or 1a, wherein the isolated cells are selected from podocytes or tubular epithelial cells.
Embodiment 3 is the method of embodiment 2, wherein the sample is a urine sample and the isolated cells are podocytes.
Embodiment 4 is the method of embodiment 3, wherein the podocytes are isolated from the urine sample by a podocyte specific antibody.
Embodiment 5 is the method of embodiment 4, wherein the podocyte specific antibody is selected from an anti-nephrin antibody, an anti-podocin antibody, and/or an anti-synaptopodin antibody.
Embodiment 6 is the method of any one of embodiments 1 to 5, wherein the level of expression of CaMK4, IL-23, IL-17 receptor, and/or arginase 1 is determined using a reverse transcriptase-polymerase chain reaction (RT-PCR), a quantitative RT-PCR (qRT-PCR) assay, a northern blot assay, a microarray assay, a flow cytometry assay, an immunofluorescence assay, and/or a western blot assay.
Embodiment 7 is the method of any one of embodiments 1 to 6, wherein the subject has a disease selected from glomerulonephritis, anti-neutrophilic cytoplasmic autoantibody (ANCA) vasculitis, or systemic lupus erythematosus (SLE).
Embodiment 8 is a method of monitoring the response to the treatment of a subject with an active lupus nephritis (LN) flare, the method comprising:
   a. obtaining a sample from the subject undergoing treatment for an active LN flare;
   b. isolating cells from the sample; and
   c. determining a level of expression of calcium/calmodulin dependent protein kinase IV (CaMK4), IL-23, IL-17 receptor, or arginase 1, wherein an decreased level of expression of CaMK4, IL-23, and/or IL-17 receptor as compared to a control or an increased level of expression of arginase 1 as compared to a control indicates an effective response to the treatment of the active LN flare in the subject.
Embodiment 8a is the method of embodiment 8, wherein the sample is selected from a tissue sample or a urine sample.
Embodiment 9 is the method of embodiment 8 or 8a, wherein the isolated cells are selected from podocytes or tubular epithelial cells.
Embodiment 10 is the method of embodiment 9, wherein the sample is a urine sample and the isolated cells are podocytes.
Embodiment 11 is the method of embodiment 10, wherein the podocytes are isolated from the urine sample by a podocyte specific antibody.
Embodiment 12 is the method of embodiment 11, wherein the podocyte specific antibody is selected from an anti-nephrin antibody, an anti-podocin antibody, and/or an anti-synaptopodin antibody.
Embodiment 13 is the method of embodiment 8, wherein the level of expression of CaMK4, IL-23, IL-17 receptor, and/or arginase 1 is determined using a reverse transcriptase-polymerase chain reaction (RT-PCR), a quantitative RT-PCR (qRT-PCR) assay, a northern blot assay, a microarray assay, a flow cytometry assay, an immunofluorescence assay, and/or a western blot assay.
Embodiment 14 is the method of any one of embodiments 8 to 13, wherein the subject has a disease selected from glomerulonephritis, anti-neutrophilic cytoplasmic autoantibody (ANCA) vasculitis, or systemic lupus erythematosus (SLE).
Embodiment 15 is a method for identifying and/or diagnosing an active lupus nephritis (LN) flare in a subject, the method comprising:
   a. obtaining a urine sample from the subject;
   b. isolating podocytes from the urine sample;
   c. determining a level of isolated podocytes, wherein an increased level of podocytes as compared to a control indicates an active lupus nephritis (LN) flare.
Embodiment 15a the method of embodiment 15, wherein the method further comprises (d) determining an expression level of CaMK4 in the isolated urine podocytes, wherein an increased level of expression further indicates an active lupus nephritis (LN) flare.
Embodiment 16 is the method of embodiment 15 or 15a, wherein the podocytes are isolated from the urine sample by a podocyte specific antibody.
Embodiment 17 is the method of embodiment 16, wherein the podocyte specific antibody is selected from an anti-nephrin antibody, an anti-podocin antibody, and/or an anti-synaptopodin antibody.
Embodiment 18 is the method of any one of embodiments 15 to 17, wherein the subject has a disease selected from glomerulonephritis, anti-neutrophilic cytoplasmic autoantibody (ANCA) vasculitis, or systemic lupus erythematosus (SLE).
Embodiment 19 is a method of identifying an active lupus nephritis (LN) flare in a subject, the method comprising:
   a. culturing a podocyte cell line;
   b. obtaining serum IgG from the subject;
   c. contacting the podocyte cell line with the serum IgG;
   d. determining a level of expression of calcium/calmodulin dependent protein kinase IV (CaMK4), IL-17 receptor, IL-23, and/or CD86 in the IgG-contacted podocyte cell line, wherein an increase in the level of expression of CaMK4, IL-17 receptor, IL-23, and/or CD86 as compared to a control indicates an active lupus nephritis (LN) flare in the subject.
Embodiment 20 is the method of embodiment 19, wherein the level of expression of CaMK4, IL-17 receptor, IL-23, and/or CD86 is determined using a reverse transcriptase-polymerase chain reaction (RT-PCR), a quantitative RT-PCR (qRT-PCR) assay, a northern blot assay, a microarray assay, and/or a western blot assay.
Embodiment 21 is the method of embodiment 19, further comprising determining a level of CD80, tumor necrosis factor-alpha (TNF-α), and/or IL-6, wherein an increase in the level of expression of CaMK4, IL-17 receptor, IL-23, and/or CD86 and no change in the level of expression of CD80, TNF-α, and/or IL-6 as compared to a control further indicates an active LN flare in the subject.
Embodiment 22 is the method of embodiment 21, wherein the level of expression of CD80, TNF-α, and/or CD86 is determined using a reverse transcriptase-polymerase chain reaction (RT-PCR), a quantitative RT-PCR (qRT-PCR) assay, a northern blot assay, a microarray assay, and/or a western blot assay.
Embodiment 23 is the method of any one of embodiments 19 to 22, wherein the subject a disease selected from glomerulonephritis, anti-neutrophilic cytoplasmic autoantibody (ANCA) vasculitis, or systemic lupus erythematosus (SLE).
Embodiment 24 is a method of monitoring the response to the treatment of a subject with an active lupus nephritis (LN) flare, the method comprising:
   a. culturing a podocyte cell line;
   b. obtaining serum IgG from the subject undergoing treatment for an active LN flare;
   c. contacting the podocyte cell line with the serum IgG;
   d. determining a level of expression of calcium/calmodulin dependent protein kinase IV (CaMK4), IL-17 receptor, IL-23, and/or CD86 in the IgG-contacted podocyte cell line, wherein a decrease in the level of expression of CaMK4, IL-17 receptor, IL-23, and/or CD86 as compared to a control indicates an effective response to the treatment of the active LN flare in the subject.
Embodiment 25 is the method of embodiment 24, wherein the level of expression of CaMK4, IL-17 receptor, IL-23, and/or CD86 is determined using a reverse transcriptase-polymerase chain reaction (RT-PCR), a quantitative RT-PCR (qRT-PCR) assay, a northern blot assay, a microarray assay, and/or a western blot assay.
Embodiment 26 is the method of embodiment 24, further comprising determining a level of CD80, tumor necrosis factor-alpha (TNF-α), and/or IL-6, wherein an increase in the level of expression of CaMK4, IL-17 receptor, IL-23, and/or CD86 and no change in the level of expression of CD80, TNF-α, and/or IL-6 as compared to a control further indicates an active LN flare in the subject.
Embodiment 27 is the method of embodiment 26, wherein the level of expression of CD80, TNF-α, and/or CD86 is determined using a reverse transcriptase-polymerase chain reaction (RT-PCR), a quantitative RT-PCR (qRT-PCR) assay, a northern blot assay, a microarray assay, and/or a western blot assay.
Embodiment 28 is the method of any one of embodiments 24 to 27, wherein the subject a disease selected from glomerulonephritis, anti-neutrophilic cytoplasmic autoantibody (ANCA) vasculitis, or systemic lupus erythematosus (SLE).

### EXAMPLES

### Example 1: Aberrantly Glycosylated IgG Elicited Pathogenic Signaling in Podocytes and Signified Lupus Nephritis (LN)

### Patients and Methods

Patients and controls: 30 patients (18-65 years old), who were referred to the center for kidney biopsy between 2017-2019 to evaluate the presence of lupus nephritis (LN) were studied. De-identified clinical and pathologic information was extracted from biopsy reports for patients whose biopsies were included. Five tissue samples were obtained from individuals who underwent a kidney biopsy but had no identifiable glomerular lesion. The urine and serum of 15 additional patients, who fulfilled at least 4 of the 11 American College of Rheumatology revised criteria for the classification of systemic lupus erythematosus (SLE), was also analyzed. All patients were women between the ages of 20 and 64 years and had SLE disease activity index scores ranging from 8 to 16. Serum samples were collected and stored at −80° C. until used. Fresh urine was collected and evaluated as specified below

Study approval: Human kidney biopsies were collected at the Beth Israel Deaconess Medical Center. The protocol concerning the use of biopsy, serum, and urine samples from patients with LN was approved by the Institutional Review Boards on human subjects at Beth Israel Deaconess Medical Center (no. 088-2015).

IgG purification. IgG purification kits (Dojindo Molecular Technologies; Rockville, MD) were used to purify IgG from SLE patients or healthy volunteers according to the manufacturer's protocol. Purity was confirmed by SDS-PAGE.

Immortalized human podocyte cell line: The immortalized human podocyte cell line was cultured as previously described (16,39). Briefly, cells were cultured with RPMI-1640 with 10% FBS, insulin, transferrin, and selenium. These cells proliferate at 33° C. and differentiate into mature podocytes for 7 to 10 days after transfer to 37° C. due to the temperature-sensitive SV40-T gene and a telomerase gene. Ten days after being transferred to 37° C., cells were treated with IgG (10 μg/mL) from patients with SLE with active LN, SLE without kidney involvement, or healthy controls. These cells were collected 24 to 72 hours after stimulation and analyzed by Western blotting.

Western blotting: Podocytes were lysed in RIPA buffer or NP40 lysis buffer at 4° C. for 30 minutes. After centrifugation (16,400 g; 30 minutes; 4° C.), supernatants were collected and an identical amount of protein from each lysate was separated on NuPAGE 4%-12% Bis-Tris Gel (Thermo Fisher). Proteins were transferred to a nitrocellulose membrane, which was subsequently blocked for 1 hour using 5% nonfat dry milk or 3% BSA in TBS-T and incubated at 4° C. overnight with mouse anti-human CAMK4 (catalog 610276/clone 26, BD Biosciences; San Jose, CA), NFκB p65(CST D14E12), nephrin, mouse anti human SNAIL(CST L70G2) and mouse anti-human GAPDH (catalog 649202/FF26A/F9, BD Biosciences). The membrane was washed with TBS-T and incubated with anti-rabbit or anti-mouse, IgG coupled with HRP (catalog sc-2004/sc-2020/sc-2005/sc-2473, Santa Cruz Biotechnology Inc.; Dallas, TX). The ECL system (Amersham; Little Chalfont, United Kingdom) was used for detection. Bands on blots corresponding to proteins of interest were analyzed by ImageJ software (NIH; Bethesda, MD).

Real-time PCR: Total mRNA was isolated from human podocytes or soiled urine podocytes using the RNeasy Mini Kit (QIAGEN; Hilden, Germany), and then cDNA was synthesized using cDNA EcoDry Premix (Clontech; Mountain View, CA) for PCR amplification. Real-time PCR analysis was performed with the Light Cycler 480 System (Roche; Basel, Switzerland) using TaqMan gene expression assays according to the manufacturer's specifications (Applied Biosystems; Foster City, CA). Expression was normalized to GAPDH. All primers and probes were from Applied Biosystems and were as follows: CaMK4 (Mm01135329 ml), NPHS1 (Hs001 90446 ml), and GAPDH (Hs02786624_g1). Gene expression was assessed by the comparative Ct method.

Transfecting with small interfering RNA (siRNA): Human podocytes were transfected with CaMK4 siRNA, FCRN siRNA, NFκB p65, or control siRNA (Thermo Scientific, CST; Waltham, MA) using INTERFERin transfection reagent (Polvplus Transfection; New York, NY) according to the manufacturer's protocol. After 24 or 48 hours of incubation, the cells were exposed to IgG and then collected for RNA extraction or were stained for immunofluorescence analysis.

Immunofluorescence: Frozen kidney sections (4 μm) were fixed in 4% formaldehyde for 10 minutes or acetone for 3 minutes and blocked for 1 hour in BSA, followed by overnight incubation with mouse anti-human CaMK4 antibody (catalog 610276/clone 26, BD Biosciences), or goat anti-human synaptopodin antibody. Next, sections were washed and stained for 1 hour with Alexa Fluor 488- or 568-labeled donkey anti-goat or anti-mouse antibodies. Finally, DAPI or Hoechst 33258 (Life Technologies; Carlsbad, CA) was applied for nuclear staining.

Cultured podocytes (0.2×10⁵) were seeded onto type I collagen 4-well culture slides (BD Biosciences) and exposed for 24 hours to fluorescence-labeled IgG or non-labeled IgG (10 g/mL) from LN patients and healthy individuals. After 30 minutes of incubation with RPMI 1640 medium supplemented with 2% bovine serum albumin (BSA), the cells were washed once with PBS and fixed for 20 minutes with 4% paraformaldehyde. The cells were permeabilized for 5 minutes with 0.1% Triton X-100 in PBS, followed by blocking for 30 minutes with PBS containing 2% BSA. Cells were then stained for 1 hour at room temperature with anti-FcRn antibody (1:100 dilution; Santa Cruz Biotechnology). After washing 3 times with PBS, the cells were stained with Alexa Fluor 568 anti-rabbit IgG (highly cross-absorbed) as the secondary antibody (Invitrogen; Carlsbad, CA). The stained cover glasses were mounted on a glass slide with 10 μL of DAPI Fluoromount-G (Southern Biotech; Birmingham, AL) and sealed with nail polish.

Stained specimens were analyzed with a Nikon Eclipse Ti confocal microscope. Images were analyzed with EZ-C1 v.3.7 software, and fluorescence intensities or areas were measured by ImageJ software.

Cell isolation from urine: Urine was spun at 1500 rpm for 5 minutes and the supernatant was discarded. PBS was used to resuspend the sediment and then exposed to antibody-coated magnetic beads with rotation for 10 minutes at room temperature. The antibodies that were used were directed against three podocyte-specific proteins nephrin, podocin, and synaptopodin. A magnet was used to separate the cells (podocytes) from the beads. Cells were isolated from beads.

Deglycosylation of IgG: PNGase F, α-fucosidase, neuraminidases S and A, and β-galactosidase were purchased from New England Biolabs (Ipswich, MA) and deglycosylation was performed following the manufacturer's protocol without denaturing the protein.

Mass spectrometry analysis of IgG N-glycans: Approximately 7 pug of purified IgG of each sample were loaded into an SDS-page gel (4-12%). After staining with Coomassie Brilliant Blue, the bands corresponding to the IgG heavy chain (˜50 kDa) were excised. The gel pieces were washed with a solution of 50% acetonitrile in 50 mM ammonium bicarbonate, briefly dried with a vacuum centrifuge, and incubated with 200 μL of 10 mM 1,4-Dithiothreitol (DTT) for 30 minutes at 50° C. The DTT solution was then discarded and the gel pieces were washed with acetonitrile and briefly dried. 200 μL of 55 mM iodoacetamide (IAA) were added to the samples and incubated 30 minutes at RT in the dark. The IAA solution was next discarded, the samples were washed with 50 mM ammonium bicarbonate (AMBIC) followed with acetonitrile before briefly drying the gel pieces. The samples were then incubated with 500 μL of a TPCK-treated trypsin solution (20 μg/mL in 50 mM AMBIC) at 37° C. overnight. The supernatants were recovered in new tubes before carrying out two sequential washes with 200 μL of 50 mM AMBIC, vortexed for 15 min; 200 μL of 50% acetonitrile in 50 mM AMBIC, vortexed for 15 minutes; and 200 μL of acetonitrile, vortexed for 15 minutes. For each sample, all washes were collected, pooled in the same tube and lyophilized.

The dried materials were resuspended in 200 μL of 50 mM AMBIC to which 1 μL of PNGase F was added for an overnight incubation at 37° C. The released N-glycans were purified over a C18 Sep-Pak (50 mg) column (Waters, Milford, MA, USA) conditioned beforehand with 1 column volume (CV) of methanol, 1 CV of 5% acetic acid, 1 CV of 1-propanol, and 1 CV of 5% acetic acid. The C18 column was washed with 5% acetic acid, flow through; and wash fractions were collected, pooled, and lyophilized.

Lyophilized N-glycan samples were incubated with 1 mL of a DMSO-NaOH slurry solution and 500 μl of methyl iodide for 30 minutes under vigorous shaking at RT. 1 mL of Milli-Q water was then added to stop the reaction followed by 1 mL of chloroform to purify the permethylated N-glycans. The chloroform fractions were washed three times with 3 mL of Milli-Q water. The chloroform fractions were dried before being re-dissolved in 200 mL of 50% methanol and was then loaded into a conditioned C18 Sep-Pak (50 mg) column with 1 CV of methanol, 1 CV of Milli-Q water, 1 CV of acetonitrile, and 1 CV of Milli-Q water. The C18 columns were washed with 3 mL of 15% acetonitrile and then eluted with 3 mL of 50% acetonitrile. The eluted fractions were lyophilized and then re-dissolved in 10 μL of 75% methanol, from which 1 μL was mixed with 1 μL DHB (2,5-dihydroxybenzoic acid) (5 mg/mL in 50% acetonitrile with 0.1% trifluoroacetic) and spotted on a MALDI polished steel target plate (Bruker Daltonics, Bremen, Germany).

MS data were acquired on a Bruker UltraFlex II MALDI-TOF Mass Spectrometer instrument. The reflective positive mode was used, and data were recorded between 500 and 6000 m/z. For each MS N-glycan profile, the aggregation of 20,000 laser shots or more were considered for data extraction. Only MS signals matching an N-glycan composition were considered for further analysis. Subsequent MS post-data acquisition analysis was made using mMass (32). The relative abundance of each N-glycans identified was calculated based on the absolute intensity of the first isotopic peak of a given N-glycan relative to the sum of all N-glycan intensities.

Statistics: Statistical analyses were performed with GraphPad Prism version 7.0 software and STATA version 15. Statistical significance was determined by t test (2-tailed) for 2 groups, 1-way ANOVA with Bonferroni multiple comparisons tests, or 2-way ANOVA with Bonferroni's multiple comparisons tests for 3 or more groups. P<0.05 was considered statistically significant. For kidney biopsy data, the descriptive characteristic of the examined population of patients was prepared, determining minimum, maximum mean, and median values. The study variables were analyzed using the logistic regression model. The model facilitates the examination of the impacts of multiple independent variables on a binary dependent variable Y. The values of variable Y were coded as follows: 1: presence of a particular trait and 0: absence of a particular trait. Correlation between the presence of LN and CaMK4 in podocytes was determined by Pearson correlation test.

### Results

IgG from SLE Patients with LN, but not without LN, Increased CaMK4 Expression in Podocytes

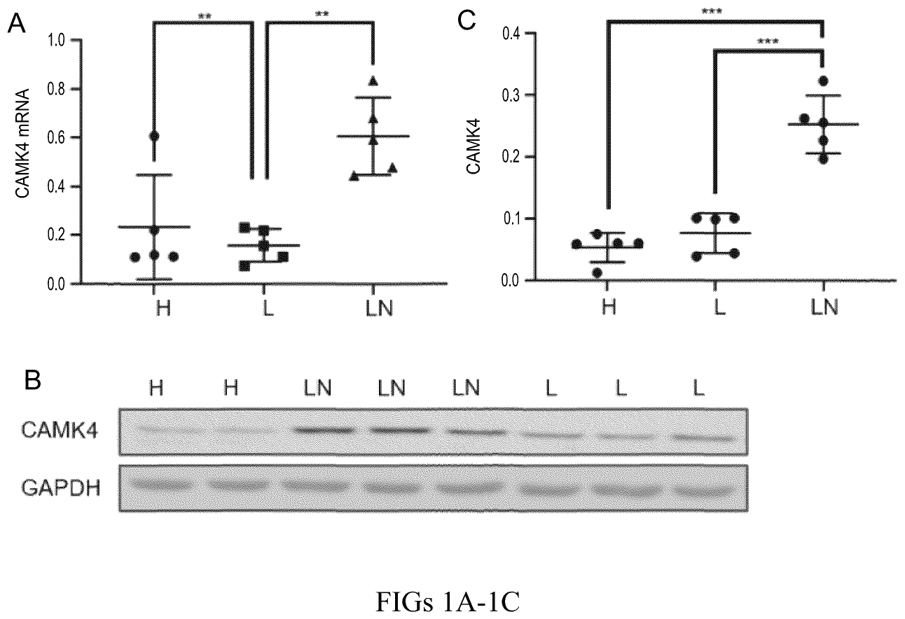

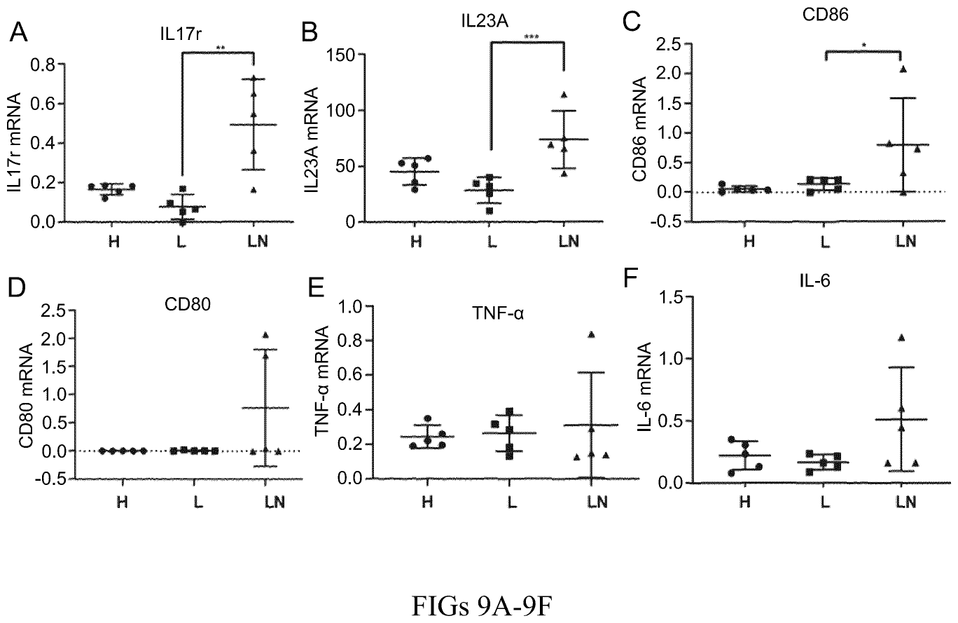

Previously, it was demonstrated that IgG from patients with SLE, but not from healthy subjects, causes upregulation of CaMK4 in podocytes followed by podocyte injury (16,19). Because not all patients with SLE develop LN (9), it was sought to evaluate whether IgG from SLE patients without evidence of LN can upregulate CaMK4 in a similar manner. To this end, cultured human podocytes were exposed to IgG from healthy subjects or SLE patients with and without LN. Fifteen samples (5 per group) from age and sex matched individuals were used (Table 1). It was found that CaMK4 expression at the protein and mRNA levels increased in podocytes cultured in the presence of IgG from patients with active LN, but not in podocytes exposed to IgG from healthy controls or SLE patients without nephritis (FIGS. 1A-1C). In parallel, it was noted that the expression of the IL17 receptor and IL-23A was increased in podocytes exposed to IgG from patients with LN, but not from SLE patients without clinical nephritis and healthy controls. The expression of the costimulatory molecule CD86, but not of CD80, followed the same pattern, whereas the expression of TNF alpha and IL-6 was not affected significantly (FIGS. 9A-9F). These data demonstrated that IgG from SLE patients with LN, but not from those without LN, induced the expression of genes which have been linked to the pathogenesis of LN (33-42).

TABLE 1
Characteristics of individuals with SLE and healthy
controls at the time of IgG isolation.

| | | | | | Urine | Urine | | Serum | |
|---|---|---|---|---|---|---|---|---|---|
| Sample | SLE | Age | Sex | LN | RBC/phpf | Protein (g) | dsDNA | Creatinine | Race |
| 1 | + | 47 | F | + | 5 | 2 | 1:640 | 1 | White |
| 2 | + | 27 | F | + | 1 | 0.7 | 1:1280 | 0.7 | Black |
| 3 | + | 22 | F | + | 1 | 0.8 | 1:1280 | 1.6 | Black |
| 4 | + | 29 | F | + | 2 | 1.7 | 1:1280 | 0.6 | Black |
| 5 | + | 40 | F | + | 0 | 6.8 | 1:10 | 0.5 | Asian |
| 6 | + | 46 | F | − | 2 | 0.1 | negative | 0.8 | White |
| 7 | + | 27 | F | − | 1 | 0 | negative | 0.9 | Black |
| 8 | + | 22 | F | − | 1 | 0.1 | negative | 0.8 | Black |
| 9 | + | 34 | F | − | 1 | 0.1 | negative | 1.3 | Black |
| 10 | + | 31 | F | − | 0 | 0.1 | negative | 0.8 | Asian |
| 11 | − | 45 | F | NA | | | | | White |
| 12 | − | 28 | F | NA | | | | | Black |
| 13 | − | 27 | F | NA | | | | | Black |
| 14 | − | 31 | F | NA | | | | | Black |
| 15 | − | 42 | F | NA | | | | | Asian |

### Fucose Enhanced, Whereas Galactose Decreased the Ability of IgG to Upregulate CaMK4

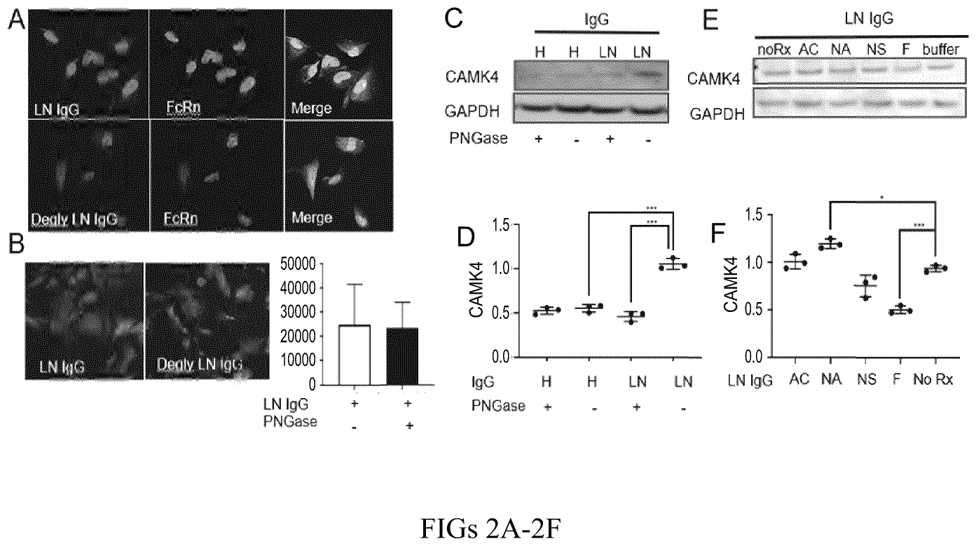

Because N-glycans regulate effector functions of IgG (43-44), it was hypothesized that modifications in IgG glycosylation were responsible for its ability to upregulate CaMK4 in podocytes. To address this hypothesis, N-linked glycans on IgG from healthy individuals and patients with active LN were enzymatically removed and their ability to increase CaMK4 expression in podocytes was evaluated. In a previous study, it had been demonstrated that IgG from individuals with SLE upregulates CaMK4 after it enters podocytes using the neonatal Fc receptor (FcRn) (29). Hence, it was first established that de-glycosylated IgG binds to FcRn (FIG. 2A) and enters the podocyte at amounts equivalent to those of non-modified IgG (FIG. 2B). In contrast to the non-modified IgG, deglycosylated IgG from patients with LN did not cause upregulation of CaMK4 in podocytes (FIGS. 2C and 2D), suggesting that upregulation of CaMK4 requires the presence of N-glycans on SLE IgG. Removal of N-glycans from normal IgG did not affect the expression of CAMK4 in exposed podocytes.

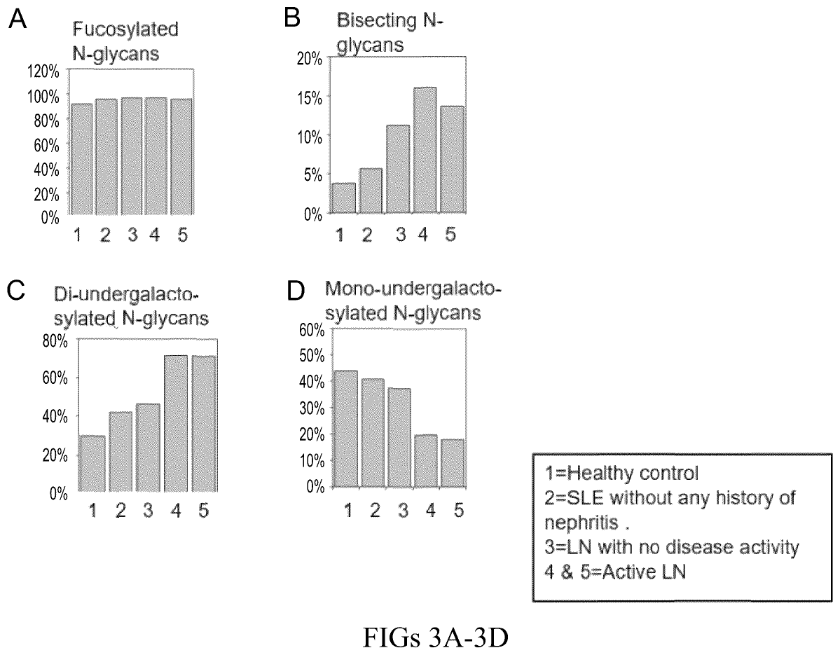

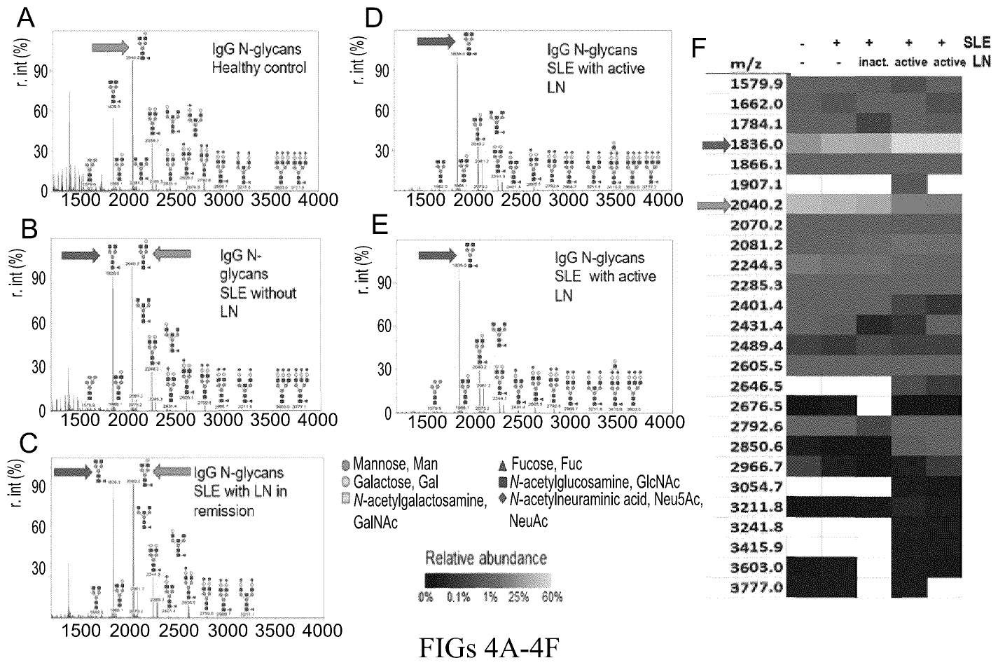

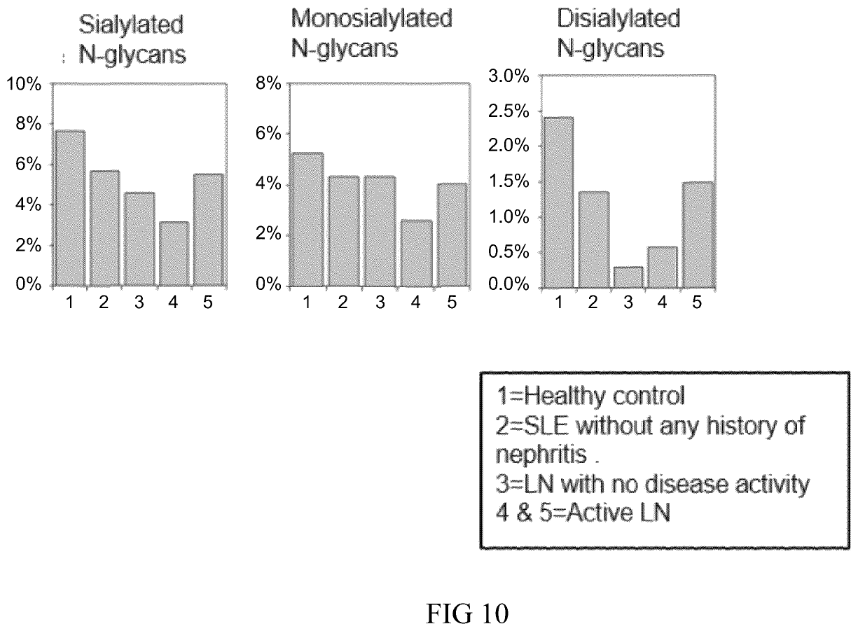

To further dissect the role of specific glycan residues, IgG from patients with LN was treated with specific glycosidases. It was found that removal of fucose following treatment with α-fucosidase abrogated the ability of IgG to upregulate CaMK4 (FIGS. 2E and 2F). To obtain additional details on N-glycan changes in IgG from individuals with LN, the IgG N-glycome of healthy controls, individuals with SLE with and without nephritis, and those with a history of LN without active clinical disease after treatment, was analyzed and compared using mass spectrometry. Surprisingly, the relative abundance of fucosylated N-glycans was similar among all samples (FIG. 3A). IgG from individuals with nephritis showed an increase in relative abundance of bisecting N-glycans, non-galactosylated N-glycans, and a decrease in relative abundance of mono-galactosylated N-glycans compared to healthy individuals and patients with SLE without active LN (FIGS. 3B-3D). There was no significant difference noted in sialylated glycans amongst the samples (FIG. 10). Unlike patients with no kidney involvement and healthy controls (FIGS. 4A-4C), the most abundant N-glycan (˜57%) on IgG from individuals with active LN was a non-galactosylated, core-fucosylated N-glycan (1836.0 m/z—red arrow) (FIGS. 4D-4F). In addition, the mono-galactosylated, core-fucosylated N-glycan (2040.2 m/z-green arrow) was found at a low abundance (˜17%) on IgG from patients with active LN (FIGS. 4D-4F). By contrast, that same mono-galactosylated, core-fucosylated N-glycan (2040.2 m/z—green arrow) was found to be most abundant (˜42%) in healthy individuals (FIGS. 4A and 4F). Interestingly, individuals with SLE and LN in remission or without any kidney involvement showed a relatively moderate abundance (˜37%) of the mono-galactosylated, core fucosylated N-glycan (2040.2 m/z—green arrow) (FIGS. 4B-4C and 4F).

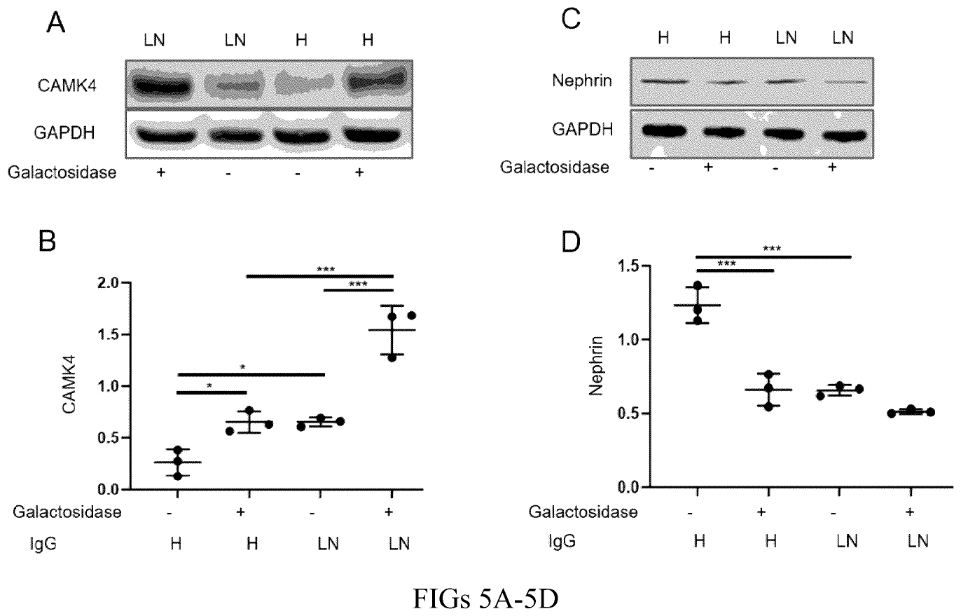

These findings demonstrated that patients with active LN have decreased IgG galactosylation. More importantly, they show that patients with active LN differ from healthy controls and patients without active renal disease in having a relatively lower abundance of IgG carrying mono-galactosylated N-glycans. It was considered that while fucose may be responsible for podocyte injury, galactose may have a protective effect and prevent CAMK4 upregulation. To address this possibility, CAMK4 expression in podocytes exposed to IgG from healthy individuals and patients with active LN before and after treatment with β1,4-galactosidase to remove the terminal galactose residue was studied. It was found that after β-galactosidase treatment, IgG from healthy controls gained the ability to upregulate CaMK4 (FIGS. 5A and 5B). Furthermore, IgG from individuals with LN displayed an enhanced ability to cause CaMK4 upregulation after treatment of IgG with β-galactosidase (FIGS. 5A and 5B).

To determine whether IgG treatment with β-galactosidase can cause podocyte injury, the expression of nephrin, a podocyte slit diaphragm protein, the downregulation of which is considered a pathologic feature of glomerular injury and podocyte viability (72) was investigated. It was found that nephrin expression was downregulated in podocytes after exposure to β-galactosidase-treated IgG from healthy controls compared to untreated IgG and reached levels comparable to those in podocytes exposed to IgG from patients with LN (FIGS. 5C and 5D). This set of experiments demonstrated that in patients with LN, IgG is aberrantly glycosylated, specifically, it lacks a terminal galactose and induces upregulation of CAMK4 to podocytes through its fucosylated core.

### CaMK4 Regulates Nephrin Transcription Through NFκB/p65 Phosphorylation and Enhanced SNAIL Transcription

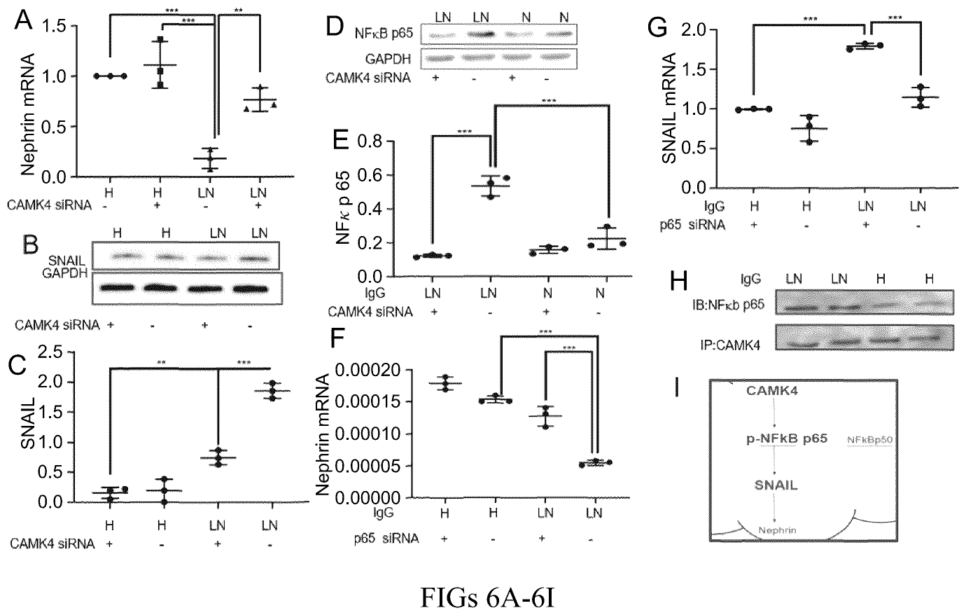

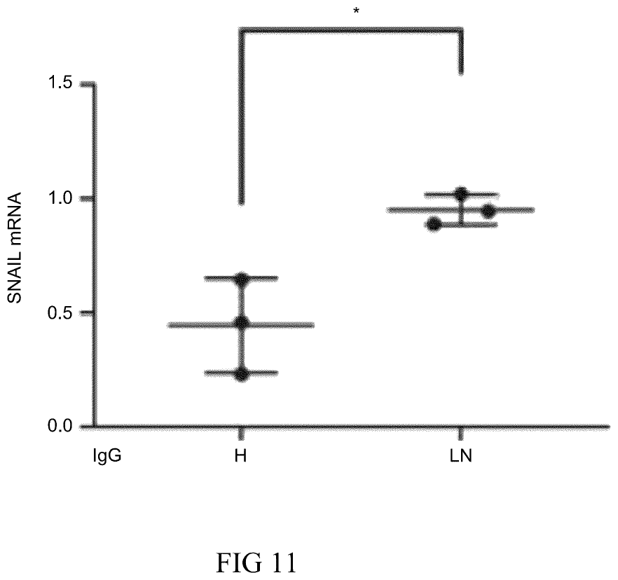

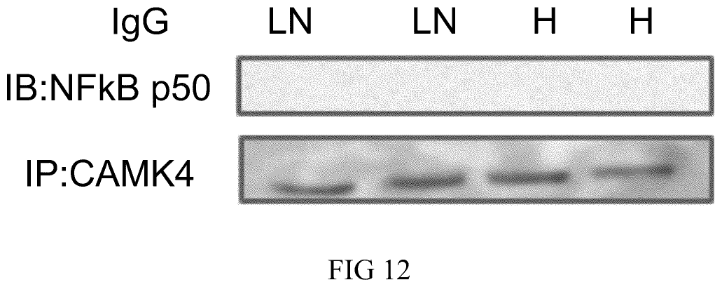

Because it had previously been shown that CaMK4 suppressed nephrin protein and mRNA expression (19), a set of experiments to define the mechanisms involved were conducted. Using siRNA to silence CaMK4, it was found that nephrin transcription was preserved in podocytes exposed to IgG from patients with LN (FIG. 6A). Because SNAIL is a canonical repressor of nephrin transcription (45), it was evaluated whether CaMK4 downregulates nephrin transcription through SNAIL. It was found that SNAIL protein and mRNA expression increased upon exposure of podocytes to IgG from patients with LN in a CaMK4-dependent manner as its expression failed to increase when CaMK4 was silenced (FIGS. 6B, 6C, and FIG. 11). Since SNAIL transcription is regulated by NFκB (46), which is phosphorylated by CAMK4 (47), a possible interaction of CaMK4 and NFκB was explored. NFκB consists of the p65 (RelA) subunit, which contains a transactivation domain responsible for the transcriptional activity, and the p50 subunit, which does not contain a transactivation domain and in its homodimeric form acts as a transcriptional repressor (48). It was found that the levels of p65 increased in podocytes after exposure to IgG from individuals with LN and decreased in the presence of CaMK4 siRNA (FIGS. 6D and 6E). It was also observed that upon silencing NFκB p65, nephrin transcription was preserved (FIG. 6F) and SNAIL levels did not increase in podocytes exposed to IgG from patients with LN (FIG. 6G). CaMK4 in podocytes was further immunoprecipitated after exposure to normal or LN IgG, and the immunoprecipitates were blotted with p65 and p50 antibodies. It was observed that while p65 interacted with CaMK4 (FIG. 6H), no significant interaction was noted with the p50 subunit (FIG. 12). These experiments revealed a pathway whereby IgG from LN upregulates CaMK4, which suppresses nephrin transcription through activation of NFκB and upregulation of the nephrin transcriptional repressor SNAIL (FIG. 6I).

### CaMK4 Expression in Renal Biopsies Identified Active LN

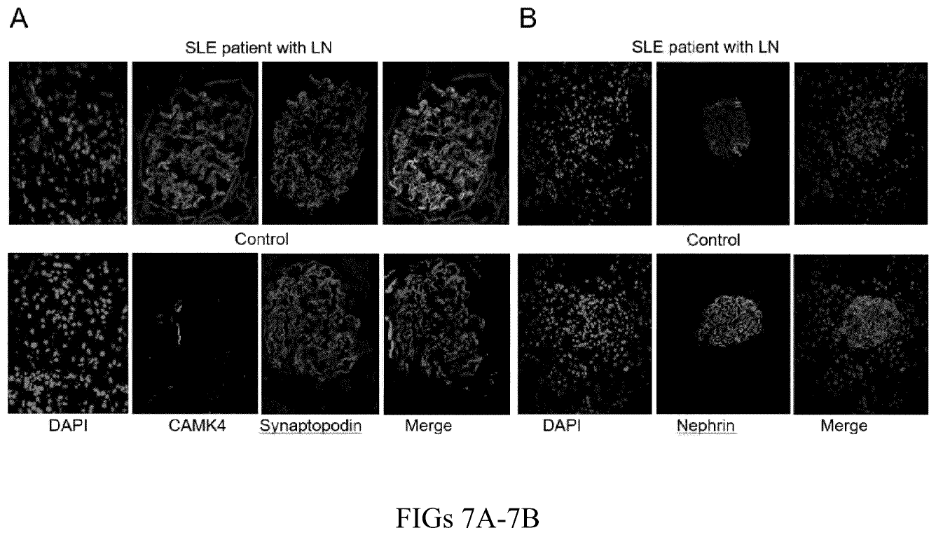

The in vitro data revealed a podocyte-specific pathogenic phenotype, which is elicited by aberrantly glycosylated IgG from patients with LN and characterized by increased expression of CaMK4 and decreased expression of nephrin. To evaluate whether this phenotype was associated with glomerular disease in patients with SLE, the expression of CaMK4 and nephrin was examined in kidney biopsies from 30 patients referred to our center for suspected LN between 2017-2018. The baseline clinical characteristics of the studied patients are listed in Table 2. CaMK4 expression was increased in the glomeruli of patients with LN. Confocal immunofluorescence studies showed that CaMK4 colocalized with synaptopodin, a podocyte marker (49), demonstrating its presence in podocytes (FIG. 7A). Consistent with the experimental data, the podocyte protein nephrin was decreased in CaMK4-overexpressing glomeruli from kidney biopsies of patients with LN compared to those of individuals who had undergone a kidney biopsy but had no identifiable glomerular lesion (control) (FIG. 7B).

TABLE 2
Characteristics of individuals referred for a kidney biopsy.

| Variable | Mean (SD)/Percentage |
|---|---|
| Age | 40.19(14.2) |
| Female | 100% |
| Duration of disease | 5.5(7.8) |
| Immunosuppression | 48.3% |
| Activity Index | 5.25(4.44) |
| Chronicity Index | 3(2.9) |

To determine whether the presence of CaMK4 in podocytes was associated with the histologic diagnosis of LN, two different logistic regression analyses were performed. Age, duration of disease, and use of immunosuppressive drugs was adjusted for first, and it was found that the presence of CaMK4 in podocytes predicted the presence of histologically proven LN (p<0.001). Next, activity and chronicity indices were adjusted for, and it was found that CAMK4 expression in podocytes was again significantly associated with the presence of LN (p=0.005). CaMK4 expression was high in 27 of the 30 specimens of individuals who had been evaluated for suspicion of LN. One of the three CaMK4-negative specimens had a diagnosis of ANCA-associated vasculitis. The two other specimens had histologic evidence of LN Class V and Class VI without evidence of histologic activity. Upon further correlation analysis, CaMK4 expression was observed to be associated with histologic activity index (correlation coefficient=0.5675). The correlation coefficients for the various factors considered are shown in Table 3 and the p (change per one standard deviation) values for each factor in Table 4. These data reveal the value of detecting CaMK4 in kidney biopsy tissue as a surrogate marker for the presence of active LN.

TABLE 3
Correlation coefficient between CaMK4 detection and LN

| | Age | Duration | IS | Lupus | AI | CI | CAMK4 |
|---|---|---|---|---|---|---|---|
| Age | 1.0000 | | | | | | |
| Duration | 0.3253 | 1.0000 | | | | | |
| IS | −0.4049 | 0.2023 | 1.0000 | | | | |
| Lupus | −0.1076 | 0.0960 | 0.1557 | 1.0000 | | | |
| AI | 0.1960 | −0.1926 | −0.372 | 0.4144 | 1.0000 | | |
| CI | −0.0756 | 0.1656 | 0.1163 | 0.4418 | 0.2119 | 1.0000 | |
| CAMK4 | −0.2549 | −0.3179 | 0.1386 | 0.7303 | 0.5675 | 0.2113 | 1.0000 |

IS: Immunosuppression;
AI: Activity Index;
CI: Chronicity Index

TABLE 4
β coefficients of variables used in multivariate regression analysis

| Variable | β |
|---|---|
| CAMK4 | .4955 |
| Age | .0048 |
| Duration | −.0067 |
| Immunosuppression | .00176 |
| Activity Index | −0.026 |
| Chronicity Index | −.0015 |
| Cons | .7553 |

CaMK4 Expression in Urine Podocytes Identifies Individuals with Active LN

Kidney biopsies are invasive with potential complications and difficult to repeat for monitoring of LN during treatment. Therefore, it was sought to determine whether the expression of CaMK4 in urine podocytes reflects active nephritis in patients with SLE. Urine from 15 individuals with SLE was collected. Four individuals did not have any kidney involvement, while five individuals had a histologic diagnosis of active LN without any prior treatment, and six individuals had partial or complete clinical response to treatment after being initially diagnosed with LN. LN was diagnosed by a kidney biopsy interpreted by a nephropathologist. Complete response was defined as improved proteinuria to <500 mg/day, inactive urine sediment, and a serum creatinine within 20% of baseline. Partial clinical response was defined as a 50 percent reduction in proteinuria to less than 1.5 g/day and stable serum creatinine. Clinical characteristics of these individuals are displayed in Table 5.

TABLE 5
Characteristics of Individual at the time of urine collection

| | | | | | Urine | Urine |
|---|---|---|---|---|---|---|
| | | | | Clinical | RBC/ | Protein |
| Sample | SLE | Sex | LN | Response | phpf | (g) |
| 1 | + | F | Class IV | − | 10-12 | 8 |
| 2 | + | F | Class III | − | 9 | 1.2 |
| 3 | + | F | Class IV + V | − | 20 | 1 |
| 4 | + | F | Class IV | − | 18 | 4 |
| 5 | + | F | Class V | − | 9 | 4.6 |
| 6 | + | F | — | NA | 0 | 0.1 |
| 7 | + | F | — | NA | 1 | 0.1 |
| 8 | + | F | — | NA | 0 | 0.2 |
| 9 | + | F | — | NA | 1 | 0.1 |
| 10 | + | F | Class IV | + | 2-3 | 0.5 |
| 11 | + | F | Class III + IV | + | 3 | 0.2 |
| 12 | + | F | Class II | + | 0 | 0 |
| 13 | + | F | Class III | + | 0-1 | 0 |
| 14 | + | F | Class III | + | 4 | 0.3 |
| 15 | + | F | Class IV | + | 4-5 | 1.7 |

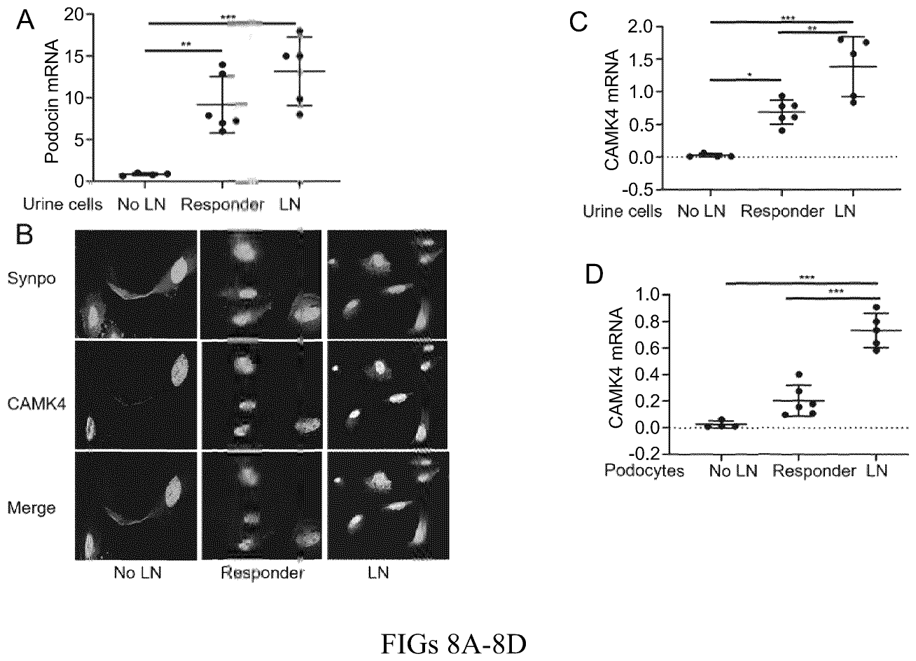

Cells in urine collected by cytospin were examined for the presence of podocytes through detection of the podocyte markers podocin by RT-PCR and synaptopodin by immunofluorescence. The data revealed that individuals with LN had a larger number of podocytes in the urine when compared to those without LN (FIG. 8A). CaMK4 staining was significantly positive in urine podocytes isolated from individuals with active LN (FIG. 8B). Furthermore, total urine cell CaMK4 mRNA expression was elevated in individuals with active LN when compared to those with clinical response post treatment and those without kidney involvement (FIG. 8C). Since the expression of CaMK4 in podocytes in kidney biopsy samples was associated with histologic diagnosis of LN, we sought to examine whether urine podocyte CaMK4 mRNA can differentiate between active and clinically inactive LN. Magnetic beads coated with antibodies to synaptopodin were used to isolate podocytes. CaMK4 mRNA levels were increased only in podocytes isolated from the urine of patients with active LN while those with clinical response had minimal CaMK4 mRNA expression (FIG. 8D). These findings supported the diagnostic value of CaMK4 in isolated urine podocytes and introduced a novel noninvasive approach to monitor LN disease activity.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All documents cited herein are incorporated by reference.

### REFERENCES

1) Yap D Y, Tang C S, Ma M K, Lam M F, Chan T M. Survival analysis and causes of mortality in patients with lupus nephritis. Nephrol Dial Transplant. 2012; 27(8):3248-3254.
2) Lerang K, Gilboe I M, Steinar Thelle D, Gran J T. Mortality and years of potential life loss in systemic lupus erythematosus: a population-based cohort study. Lupus. 2014; 23(14):1546-1552.
3) Bernatsky S, Boivin J F, Joseph L, et al. Mortality in systemic lupus erythematosus. Arthritis Rheum. 2006; 54(8):2550-2557.
4) Faurschou M, Dreyer L, Kamper A L, Starklint H, Jacobsen S. Long-term mortality and renal outcome in a cohort of 100 patients with lupus nephritis. Arthritis Care Res (Hoboken). 2010; 62(6):873-880.
5) Tektonidou M G, Dasgupta A, Ward M M. Risk of End-Stage Renal Disease in Patients With Lupus Nephritis, 1971-2015: A Systematic Review and Bayesian Meta-Analysis. Arthritis Rheumatol. 2016; 68(6):1432-1441.
6) Fox C S, Matsushita K, Woodward M, et al. Associations of kidney disease measures with mortality and end-stage renal disease in individuals with and without diabetes: a meta-analysis [published correction appears in Lancet. 2013 Feb. 2; 381(9864):374]. Lancet. 2012; 380(9854):1662-1673.
7) Mahmoodi B K, Matsushita K, Woodward M, et al. Associations of kidney disease measures with mortality and end-stage renal disease in individuals with and without hypertension: a meta-analysis [published correction appears in Lancet. 2012 Nov. 10; 380(9854):1648]. Lancet. 2012; 380(9854):1649-1661.
8) Cozzolino M, Galassi A, Pivari F, Ciceri P, Conte F. The Cardiovascular Burden in End-Stage Renal Disease. Contrib Nephrol. 2017; 191:44-57. doi:10.1159/000479250
9) Gansevoort R T, Correa-Rotter R, Hemmelgam B R, et al. Chronic kidney disease and cardiovascular risk: epidemiology, mechanisms, and prevention. Lancet. 2013; 382(9889):339-352.
10) Herzog C A, Asinger R W, Berger A K, et al. Cardiovascular disease in chronic kidney disease. A clinical update from Kidney Disease: Improving Global Outcomes (KDIGO). Kidney Int. 2011; 80(6):572-586.
11) Chen Y E, Korbet S M, Katz R S, Schwartz M M, Lewis E J; Collaborative Study Group. Value of a complete or partial remission in severe lupus nephritis. Clin J Am Soc Nephrol. 2008; 3(1):46-53.
12) Almaani S, Meara A, Rovin B H. Update on Lupus Nephritis. Clin J Am Soc Nephrol. 2017; 12(5):825-835.
13) Lech M, Anders H J. The pathogenesis of lupus nephritis. J Am Soc Nephrol. 2013; 24(9):1357-1366.
14) Couser W G. Mechanisms of glomerular injury in immune-complex disease. Kidney Int. 1985; 28(3):569-583.
15) Couser W G, Salant D J. In situ immune complex formation and glomerular injury. Kidney Int. 1980; 17(1):1-13.
16) Wener M H, Mannik M. Mechanisms of immune deposit formation in renal glomeruli. Springer Semin Immunopathol. 1986; 9(2-3):219-235.
17) Sakhi H, Moktefi A, Bouachi K, et al. Podocyte Injury in Lupus Nephritis. J Clin Med. 2019; 8(9):1340. Published 2019 Aug. 29.
18) Davidson, A. What is damaging the kidney in lupus nephritis?. Nat Rev Rheumatol 12, 143-153 (2016).
19) Maeda K, Otomo K, Yoshida N, et al. CaMK4 compromises podocyte function in autoimmune and nonautoimmune kidney disease. J Clin Invest. 2018; 128(8):3445-3459.
20) Wharram B L, Goyal M, Wiggins J E, et al. Podocyte depletion causes glomerulosclerosis: diphtheria toxin-induced podocyte depletion in rats expressing human diphtheria toxin receptor transgene. J Am Soc Nephrol. 2005; 16(10):2941-2952.
21) Wright R D, Beresford M W. Podocytes contribute, and respond, to the inflammatory environment in lupus nephritis. Am J Physiol Renal Physiol. 2018; 315(6):F1683-F1694.
22) Rosado J A, Sage S O. The actin cytoskeleton in store-mediated calcium entry. J Physiol. 2000; 526 Pt 2(Pt 2):221-229.
23) Berridge M J, Lipp P, Bootman M D. The versatility and universality of calcium signalling. Nat Rev Mol Cell Biol. 2000; 1(1):11-21.
24) Corbett E F, Michalak M. Calcium, a signaling molecule in the endoplasmic reticulum?. Trends Biochem Sci. 2000; 25(7):307-311.
25) Clapham D E. Calcium signaling. Cell. 2007; 131(6):1047-1058.
26) Berridge M J. Calcium signalling remodelling and disease. Biochem Soc Trans. 2012; 40(2):297-309.
27) Brini M, Cali T, Ottolini D, Carafoli E. Neuronal calcium signaling: function and dysfunction. Cell Mol Life Sci. 2014; 71(15):2787-2814.
28) Kaufman R J, Malhotra J D. Calcium trafficking integrates endoplasmic reticulum function with mitochondrial bioenergetics. Biochim Biophys Acta. 2014; 1843(10):2233-2239.
29) Ichinose K, Ushigusa T, Nishino A, et al. Lupus Nephritis IgG Induction of Calcium/Calmodulin-Dependent Protein Kinase IV Expression in Podocytes and Alteration of Their Function. Arthritis Rheumatol. 2016; 68(4):944-952.
30) Ichinose K, Rauen T, Juang Y T, et al. Cutting edge: Calcium/Calmodulin-dependent protein kinase type IV is essential for mesangial cell proliferation and lupus nephritis. J Immunol. 2011; 187(11):5500-5504.
31) Ichinose, K., Juang, Y.-T., Crispin, J. C., Kis-Toth, K. and Tsokos, G. C. (2011), Suppression of autoimmunity and organ pathology in lupus-prone mice upon inhibition of calcium/calmodulin-dependent protein kinase type IV. Arthritis & Rheumatism, 63: 523-529.
32) Strohalm M., Kavan D., Novak P., Volny M., Havlicek V. mMass 3: A cross-platform software environment for precise analysis of mass spectrometric data. Anal. Chem. 2010; 82:4648-4651.
33) Bhargava R, Tsokos G C. The immune podocyte. Curr Opin Rheumatol. 2019; 31(2):167-174.
34) dos Santos M, Bringhenti R N, Rodrigues P G, et al. Podocyte-associated mRNA profiles in kidney tissue and in urine of patients with active lupus nephritis. Int J Clin Exp Pathol. 2015; 8(5):4600-4613. Published 2015 May 1.
35) Aringer M, Smolen J S. Efficacy and safety of TNF-blocker therapy in systemic lupus erythematosus. Expert Opin Drug Saf. 2008; 7(4):411-419.
36) Paust, H. J. et al. The IL-23/Th17 axis contributes to renal injury in experimental glomerulonephritis. J. Am. Soc. Nephrol. 20, 969-979 (2009).
37) Aringer M, Houssiau F, Gordon C, et al. Adverse events and efficacy of TNF-alpha blockade with infliximab in patients with systemic lupus erythematosus: long-term follow-up of 13 patients. Rheumatology (Oxford). 2009; 48(11):1451-1454.
38) Soforo E, Baumgartner M, Francis L, Allam F, Phillips P E, Perl A. Induction of systemic lupus erythematosus with tumor necrosis factor blockers. Journal of Rheumatology. 2010; 37(1):204-205
39) Rovin B H, van Vollenhoven R F, Aranow C, et al. A Multicenter,
Randomized, Double-Blind, Placebo-Controlled Study to Evaluate the Efficacy and Safety of Treatment With Sirukumab (CNTO 136) in Patients With Active Lupus Nephritis. Arthritis Rheumatol. 2016; 68(9):2174-2183.
40) Odobasic D, Kitching A R, Tipping P G, Holdsworth S R. CD80 and CD86 costimulatory molecules regulate crescentic glomerulonephritis by different mechanisms. Kidney Int. 2005; 68(2):584-594.
41) Malide D, Russo P, Bendayan M. Presence of tumor necrosis factor alpha and interleukin-6 in renal mesangial cells of lupus nephritis patients. Hum Pathol. 1995; 26(5):558-564.
42) Zickert, A., Amoudruz, P., Sundstrom, Y. et al. IL-17 and IL-23 in lupus nephritis—association to histopathology and response to treatment. BMC Immunol 16, 7 (2015).
43) Anthony R M, Wermeling F, Karlsson M C, Ravetch J V. Identification of a receptor required for the anti-inflammatory activity of IVIG. Proc Natl Acad Sci USA. 2008; 105(50):19571-19578.
44) Li T, DiLillo D J, Bournazos S, Giddens J P, Ravetch J V, Wang L X. Modulating IgG effector function by Fc glycan engineering. Proc Natl Acad Sci USA. 2017; 114(13):3485-3490.
45) Matsui I, Ito T, Kurihara H, Imai E, Ogihara T, Hori M. Snail, a transcriptional regulator, represses nephrin expression in glomerular epithelial cells of nephrotic rats. Lab Invest. 2007; 87(3):273-283.
46) Tian B, Widen S G, Yang J, et al. The NFκB subunit RELA is a master transcriptional regulator of the committed epithelial-mesenchymal transition in airway epithelial cells. J Biol Chem. 2018; 293(42):16528-16545.
47) Bae J S, Jang M K, Hong S, et al. Phosphorylation of NF-kappa B by calmodulin-dependent kinase IV activates anti-apoptotic gene expression. Biochem Biophys Res Commun. 2003; 305(4):1094-1098.
48) Oeckinghaus A, Ghosh S. The NF-kappaB family of transcription factors and its regulation. Cold Spring Harb Perspect Biol. 2009; 1(4):a000034.
49) Mundel P, Heid H W, Mundel T M, Krüger M, Reiser J, Kriz W. Synaptopodin: an actin-associated protein in telencephalic dendrites and renal podocytes. J Cell Biol. 1997; 139(1):193-204.
50) Maverakis E, Kim K, Shimoda M, et al. Glycans in the immune system and The Altered Glycan Theory of Autoimmunity: a critical review. J Autoimmun. 2015; 57:1-13.
51) New J S, King R G, Kearney J F. Manipulation of the glycan-specific natural antibody repertoire for immunotherapy. Immunol Rev. 2016; 270(1):32-50.
52) Dekkers G, Rispens T, Vidarsson G. Novel Concepts of Altered Immunoglobulin G Galactosylation in Autoimmune Diseases. Front Immunol. 2018; 9:553. Published 2018 Mar. 19.
53) Schwab I, Nimmerjahn F. Role of sialylation in the anti-inflammatory activity of intravenous immunoglobulin—F(ab′)₂ versus Fc sialylation. Clin Exp Immunol. 2014; 178 Suppl 1(Suppl 1):97-99.
54) Ogata S, Shimizu C, Franco A, et al. Treatment response in Kawasaki disease is associated with sialylation levels of endogenous but not therapeutic intravenous immunoglobulin G. PLoS One. 2013; 8(12):e81448. Published 2013 Dec. 6.
55) Vučković F, Krištić J, Gudelj I, et al. Association of systemic lupus erythematosus with decreased immunosuppressive potential of the IgG glycome. Arthritis Rheumatol. 2015; 67(11):2978-2989.
56) van de Bovenkamp F S, Derksen N I L, Ooijevaar-de Heer P, et al. Adaptive antibody diversification through N-linked glycosylation of the immunoglobulin variable region. Proc Natl Acad Sci USA. 2018; 115(8):1901-1906.
57) Irvine E B, Alter G. Understanding the role of antibody glycosylation through the lens of severe viral and bacterial diseases. Glycobiology. 2020; 30(4):241-253.
58) Akilesh S, Huber T B, Wu H, et al. Podocytes use FcRn to clear IgG from the glomerular basement membrane. Proc Natl Acad Sci USA. 2008; 105(3):967-972.
59) Wilhelm I, Levit-Zerdoun E, Jakob J, et al. Carbohydrate-dependent B cell activation by fucose-binding bacterial lectins. Sci Signal. 2019; 12(571):eaao7194. Published 2019 Mar. 5.
60) Manabe Y, Marchetti R, Takakura Y, et al. The Core Fucose on an IgG Antibody is an Endogenous Ligand of Dectin-1. Angew Chem Int Ed Engl. 2019; 58(51):18697-18702.
61) Lauc G, Huffman J E, Pučić M, Zgaga L, Adamczyk B, Mužinić A, et al. (2013) Loci Associated with N-Glycosylation of Human Immunoglobulin G Show Pleiotropy with Autoimmune Diseases and Haematological Cancers. PLoS Genet 9(1): e1003225.
62) Emancipator S N, Iida K, Nussenzweig V, Gallo G R. Monoclonal antibodies to human complement receptor (CR1) detect defects in glomerular diseases. Clin Immunol Immunopathol. 1983; 27(2):170-175.
63) Kazatchkine M D, Fearon D T, Appay M D, Mandet C, Bariety J. Immunohistochemical study of the human glomerular C3b receptor in normal kidney and in seventy-five cases of renal diseases: loss of C3b receptor antigen in focal hyalinosis and in proliferative nephritis of systemic lupus erythematosus. J Clin Invest. 1982; 69(4):900-912.
64) Moll S, Miot S, Sadallah S, Gudat F, Mihatsch M J, Schifferli J A. No complement receptor 1 stumps on podocytes in human glomerulopathies. Kidney Int. 2001; 59(1):160-168.
65) Inal J M, Hui K M, Miot S, et al. Complement C2 receptor inhibitor trispanning: a novel human complement inhibitory receptor. J Immunol. 2005; 174(1):356-366.
66) Bao L, Haas M, Quigg R J. Complement factor H deficiency accelerates development of lupus nephritis. J Am Soc Nephrol. 2011; 22(2):285-295.
67) Noris M, Remuzzi G. Overview of complement activation and regulation. Semin Nephrol. 2013; 33(6):479-492.
68) Li X, Ding F, Zhang X, Li B, Ding J. The Expression Profile of Complement Components in Podocytes. Int J Mol Sci. 2016; 17(4):471. Published 2016 Mar. 30.
69) Goldwich A, Burkard M, Olke M, et al. Podocytes are nonhematopoietic professional antigen-presenting cells. J Am Soc Nephrol. 2013; 24(6):906-916.
70) Reiser J, von Gersdorff G, Loos M, et al. Induction of B7-1 in podocytes is associated with nephrotic syndrome. J Clin Invest. 2004; 113(10):1390-1397.
71) Khullar B, Balyan R, Oswal N, et al. Interaction of CD80 with Nephl: a potential mechanism of podocyte injury. Clin Exp Nephrol. 2018; 22(3):508-516.
72) Li X, Chuang P Y, D'Agati V D, et al. Nephrin Preserves Podocyte Viability and Glomerular Structure and Function in Adult Kidneys. J Am Soc Nephrol. 2015; 26(10):2361-2377.

It is claimed:

1. A method for identifying and/or diagnosing an active lupus nephritis (LN) flare in a subject, the method comprising:
a. obtaining a sample from the subject;
b. isolating cells from the sample; and
c. determining a level of expression of calcium/calmodulin dependent protein kinase IV (CaMK4), IL-23, IL-17 receptor, or arginase 1, wherein an increased level of expression of CaMK4, IL-23, and/or IL-17 receptor as compared to a control or a decreased level of expression of arginase 1 as compared to a control indicates an active lupus nephritis (LN) flare in the subject.

2. The method of claim 1, wherein the sample is selected from a tissue sample or a urine sample.

3. The method of claim 1 or 2, wherein the isolated cells are selected from podocytes or tubular epithelial cells.

4. The method of claim 3, wherein the sample is a urine sample and the isolated cells are podocytes.

5. The method of claim 4, wherein the podocytes are isolated from the urine sample by a podocyte specific antibody.

6. The method of claim 5, wherein the podocyte specific antibody is selected from an anti-nephrin antibody, an anti-podocin antibody, and/or an anti-synaptopodin antibody.

7. The method of claim 1, wherein the level of expression of CaMK4, IL-23, IL-17 receptor, and/or arginase 1 is determined using a reverse transcriptase-polymerase chain reaction (RT-PCR), a quantitative RT-PCR (qRT-PCR) assay, a northern blot assay, a microarray assay, a flow cytometry assay, an immunofluorescence assay, and/or a western blot assay.

8. The method of any one of claims 1 to 7, wherein the subject has a disease selected from glomerulonephritis, anti-neutrophilic cytoplasmic autoantibody (ANCA) vasculitis, or systemic lupus erythematosus (SLE).

9. A method of monitoring the response to the treatment of a subject with an active lupus nephritis (LN) flare, the method comprising:
a. obtaining a sample from the subject undergoing treatment for an active LN flare;
b. isolating cells from the sample; and
c. determining a level of expression of calcium/calmodulin dependent protein kinase IV (CaMK4), IL-23, IL-17 receptor, and/or arginase 1, wherein an decreased level of expression of CaMK4, IL-23, and/or IL-17 receptor as compared to a control or an increased level of expression of arginase 1 as compared to a control indicates an effective response to the treatment of the active LN flare in the subject.

10. The method of claim 9, wherein the sample is selected from a tissue sample or a urine sample.

11. The method of claim 9 or 10, wherein the isolated cells are selected from podocytes or tubular epithelial cells.

12. The method of claim 11, wherein the sample is a urine sample and the isolated cells are podocytes.

13. The method of claim 12, wherein the podocytes are isolated from the urine sample by a podocyte specific antibody.

14. The method of claim 13, wherein the podocyte specific antibody is selected from an anti-nephrin antibody, an anti-podocin antibody, and/or an anti-synaptopodin antibody.

15. The method of any one of claims 9 to 14, wherein the level of expression of CaMK4, IL-23, IL-17 receptor, and/or arginase 1 is determined using a reverse transcriptase-polymerase chain reaction (RT-PCR), a quantitative RT-PCR (qRT-PCR) assay, a northern blot assay, a microarray assay, a flow cytometry assay, an immunofluorescence assay, and/or a western blot assay.

16. The method of any one of claims 9 to 15, wherein the subject has a disease selected from glomerulonephritis, anti-neutrophilic cytoplasmic autoantibody (ANCA) vasculitis, or systemic lupus erythematosus (SLE).

17. A method for identifying and/or diagnosing an active lupus nephritis (LN) flare in a subject, the method comprising:
a. obtaining a urine sample from the subject;
b. isolating podocytes from the urine sample;
c. determining a level of isolated podocytes, wherein an increased level of podocytes as compared to a control indicates an active lupus nephritis (LN) flare.

18. The method of claim 17, wherein the method further comprises (d) determining a level of expression of CaMK4, wherein an increased level of expression of CaMK4 as compared to a control indicates an active LN flare.

19. The method of claim 17 or 18, wherein the podocytes are isolated from the urine sample by a podocyte specific antibody.

20. The method of claim 19, wherein the podocyte specific antibody is selected from an anti-nephrin antibody, an anti-podocin antibody, and/or an anti-synaptopodin antibody.

21. The method of any one of claims 17 to 20, wherein the subject has a disease selected from glomerulonephritis, anti-neutrophilic cytoplasmic autoantibody (ANCA) vasculitis, or systemic lupus erythematosus.

22. A method of identifying an active lupus nephritis (LN) flare in a subject, the method comprising:
a. culturing a podocyte cell line;
b. obtaining serum IgG from the subject;
c. contacting the podocyte cell line with the serum IgG;
d. determining a level of expression of calcium/calmodulin dependent protein kinase IV (CaMK4), IL-17 receptor, IL-23, and/or CD86 in the IgG-contacted podocyte cell line, wherein an increase in the level of expression of CaMK4, IL-17 receptor, IL-23, and/or CD86 as compared to a control indicates an active lupus nephritis (LN) flare in the subject.

23. The method of claim 22, wherein the level of expression of CaMK4, IL-17 receptor, IL-23, and/or CD86 is determined using a reverse transcriptase-polymerase chain reaction (RT-PCR), a quantitative RT-PCR (qRT-PCR) assay, a northern blot assay, a microarray assay, and/or a western blot assay.

24. The method of claim 22, further comprising determining a level of CD80, tumor necrosis factor-alpha (TNF-α), and/or IL-6, wherein an increase in the level of expression of CaMK4, IL-17 receptor, IL-23, and/or CD86 and no change in the level of expression of CD80, TNF-α, and/or IL-6 as compared to a control further indicates an active LN flare in the subject.

25. The method of claim 24, wherein the level of expression of CD80, TNF-α, and/or CD86 is determined using a reverse transcriptase-polymerase chain reaction (RT-PCR), a quantitative RT-PCR (qRT-PCR) assay, a northern blot assay, a microarray assay, and/or a western blot assay.

26. The method of any one of claims 22 to 25, wherein the subject a disease selected from glomerulonephritis, anti-neutrophilic cytoplasmic autoantibody (ANCA) vasculitis, or systemic lupus erythematosus (SLE).

27. A method of monitoring the response to the treatment of a subject with an active lupus nephritis (LN) flare, the method comprising:
a. culturing a podocyte cell line;
b. obtaining serum IgG from the subject undergoing treatment for an active LN flare;
c. contacting the podocyte cell line with the serum IgG;
d. determining a level of expression of calcium/calmodulin dependent protein kinase IV (CaMK4), IL-17 receptor, IL-23, and/or CD86 in the IgG-contacted podocyte cell line, wherein a decrease in the level of expression of CaMK4, IL-17 receptor, IL-23, and/or CD86 as compared to a control indicates an effective response to the treatment of the active LN flare in the subject.

28. The method of claim 27, wherein the level of expression of CaMK4, IL-17 receptor, IL-23, and/or CD86 is determined using a reverse transcriptase-polymerase chain reaction (RT-PCR), a quantitative RT-PCR (qRT-PCR) assay, a northern blot assay, a microarray assay, and/or a western blot assay.

29. The method of claim 27, further comprising determining a level of CD80, tumor necrosis factor-alpha (TNF-α), and/or IL-6, wherein an increase in the level of expression of CaMK4, IL-17 receptor, IL-23, and/or CD86 and no change in the level of expression of CD80, TNF-α, and/or IL-6 as compared to a control further indicates an active LN flare in the subject.

30. The method of claim 29, wherein the level of expression of CD80, TNF-α, and/or CD86 is determined using a reverse transcriptase-polymerase chain reaction (RT-PCR), a quantitative RT-PCR (qRT-PCR) assay, a northern blot assay, a microarray assay, and/or a western blot assay.

31. The method of any one of claims 27 to 30, wherein the subject a disease selected from glomerulonephritis, anti-neutrophilic cytoplasmic autoantibody (ANCA) vasculitis, or systemic lupus erythematosus (SLE).
